(19) United States
(12) Patent Application Publication
(10) Pub. No.: US 2016/0158253 A1
(43) Pub. Date: Jun. 9, 2016

(54) TREATMENT OF PANCREATIC CANCER WITH A COMBINATION OF A HYPOXIA-ACTIVATED PRODRUG AND A TAXANE

(71) Applicants: THRESHOLD PHARMACEUTICALS, INC., South San Francisco, CA (US); MERCK PATENT GMBH, Darmstadt (DE)

(72) Inventors: CHARLES P. HART, South San Francisco, CA (US); JESSICA D. SUN, South San Francisco, CA (US); BRIAN A. ELENBAAS, Darmstadt (DE); ANTONIO GUALBERTO, Darmstadt (DE)

(21) Appl. No.: 14/907,190

(22) PCT Filed: Jul. 23, 2014
(86) PCT No.: PCT/US2014/047885

§ 371 Date: Jan. 22, 2016

Related U.S. Application Data
(60) Provisional application No. 61/994,295, filed on May 16, 2014.
(60) Provisional application No. 61/887,873, filed on Oct. 7, 2013.
(60) Provisional application No. 61/859,152, filed on Jul. 26, 2013.

(51) Int. Cl. A61K 31/675; A61K 31/337; A61K 47/48; A61K 31/7068

(52) U.S. Cl. CPC A61K 31/675; A61K 31/7068; A61K 31/337; A61K 47/48284

(57) ABSTRACT

Combined administration of a hypoxia-activated prodrug, such as TH-302, a taxane, such as nab-paclitaxel, and a nucleoside analog chemotherapeutic, such as gemcitabine, are efficacious in the treatment of cancer, including pancreatic cancer.

### RELATED APPLICATIONS

This application claims priority to U.S. provisional patent application 61/859,152, filed Jul. 26, 2013; U.S. provisional patent application 61/887,873, filed Oct. 7, 2013; and U.S. provisional patent application 61/994,295, filed May 16, 2014. The priority applications are hereby incorporated herein by reference in their entirety for all purposes.

### FIELD OF THE INVENTION

The invention relates generally to the fields of biology, chemistry, medicine, molecular biology, toxicology, and pharmacology. More particularly, it provides methods for treating cancer with a combination of a hypoxia-activated prods such as TH-302, a taxane such as nab-paclitaxel, and optionally a nucleoside chemotherapeutic such as gemcitabine,

### BACKGROUND OF THE INVENTION

Pancreatic cancer is a malignant neoplasm comprising transformed cells of pancreatic origin. About 95% of these tumors are adenocarcinoma (tumors exhibiting glandular architecture on light microscopy) that derive from pancreatic exocrine cells.

Pancreatic cancer is the fourth most common cause of cancer-related deaths in the United States and the eighth worldwide (Hariharan et al., HPB (Oxford). 2008; 10(1): 58-62). Early pancreatic cancer often does not cause symptoms, and later, the symptoms are usually nonspecific and varied. For this reason, pancreatic cancer is often not diagnosed until it is advanced. Pancreatic cancer has an extremely poor prognosis: for local disease, the 5-year survival is approximately 20%, while the median survival for locally advanced and for metastatic disease, which collectively represent over 80% of individuals, is about 10 and 6 months, respectively (National Cancer Institute; Wikipedia).

Tumors often consist of highly hypoxic subregions that are known to be resistant to chemotherapy and radiotherapy. Targeting hypoxic regions with hypoxia activated prodrugs is an emerging field of pharmaceutical development. TH-302, also known by the chemical name (2-bromoethyl)({[(2-bromoethyl)amino][(2-nitro-3-methylimidazol-4-yl)methoxy]phosphoryl})amine, is a hypoxia-targeted drug being developed for the treatment of cancer, including pancreatic cancer, by Threshold Pharmaceuticals, Inc. After administration to a cancer patient, TH-302 is reduced at its nitroimidazole group, and selectively under hypoxic conditions releases the DNA bis-alkylator bromo-isophosphoramide mustard (Br-IPM). See PCT patent publication nos. WO 07/002931, WO 08/083101, WO 10/048330, WO 12/006032, WO 12/009288, WO 12/135757, WO 12/142520, WO 13/096684, WO 13/096687, and WO 13/126539, each of which is incorporated herein in their entirety by reference for all purposes.

A randomized Phase 2 clinical trial in pancreatic ductal adenocarcinoma (PDAC) has demonstrated a significant increase in progression-free survival from the addition of TH-302 to gemcitabine (GEM) versus GEM only (Borad et al., AAR Annual Meeting 2012, Abstract LB.-121; Cancer Research: Volume 72, Issue 8, Supplement 1). Because of the dire prognosis for patients with pancreatic cancer, further advancements in pharmaceutical management of the condition are needed.

### SUMMARY OF THE INVENTION

This invention provides medicines and technology for treating cancer: in particular, the invention provides methods and pharmaceutical formulations for treating cancer by administration of a combination of a hypoxia-activated prodrug, such as TH-302; a taxane such as, but not limited to, paclitaxel and nab-paclitaxel, and, optionally, a nucleoside analog chemotherapeutic, such as gemcitabine. When used together, these three drugs can be more effective and as tolerable as any of these drugs alone or a taxane used in combination with just a nucleoside analog. In some embodiments, a nucleoside analog like gemcitabine is not co-administered, as the combination of TH-302 and a taxane such as nab-paclitaxel may have substantially the same efficacy as the three drugs together or the nab-paclitaxel and gemcitabine used in combination, with a decreased side-effect profile. The drug combinations provided by this invention are effective in the treatment of cancer, including but not limited to solid tumor cancers, such as pancreatic cancer.

Thus, one aspect of this invention relates to methods and pharmaceutical formulations for treating cancer in which a combination of pharmaceutical agents comprising a hypoxia-activated prodrug such as TH-302 and a protein-bound paclitaxel such as nab-paclitaxel, with or without a nucleoside analog chemotherapeutic, is administered to a cancer patient. The drug combination may be used for simultaneous or sequential use in the treatment of cancer. Another aspect of the invention is a method of treating cancer by administering an effective combination of pharmaceutical agents comprising a hypoxia-activated prodrug such as TH-302 and a protein-bound paclitaxel such as nab-paclitaxel, with or without a nucleoside analog chemotherapeutic. A further aspect of the invention is the use of a hypoxia-activated prodrug such as TH-302 and a protein-bound paclitaxel such as nab-paclitaxel with or without a nucleoside chemotherapeutic such as gemcitabine in the manufacture of a medicament or medicament combination for treatment of cancer.

Suitable hypoxia-activated prodrugs that can be used for this purpose include those that have a structure according to Formula (I) as described in more detail later in this disclosure.

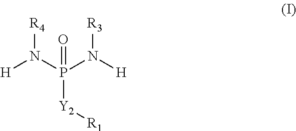

Exemplary hypoxia activated prodrugs are the hypoxia activated prodrugs TH-302 and TH-281.

Suitable taxane that can be used for this purpose include those that have a structure according to Formula (II) as described in more detail later in this disclosure.

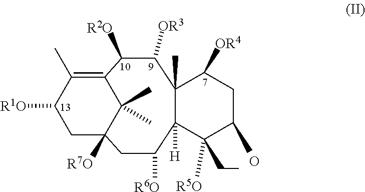

Exemplary taxane include the taxane paclitaxel. The taxane may he a protein-bound taxane and/or may be encapsulated, so as to reduce cytotoxicity or improve delivery or otherwise provide benefit. Nab-paclitaxel is an example of a protein-bound taxane.

Suitable nucleoside analog chemotherapeutics if used can include those having a structure according to Formula (III) as described in more detail later in this disclosure.

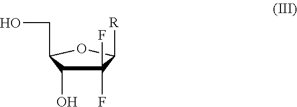

Exemplary nucleoside analogs include the nucleoside analog gemcitabine.

The hypoxia activated prodrug, the taxane, and optionally the nucleoside analog are administered at dosages and schedules such that the combination of the drugs is effective in achieving a clinically beneficial result, exemplified by but not limited to eradication or inhibition of cancer cells, stopping or slowing the rate of tumor growth, improving average life expectancy, survival, or progression-free survival, improving the quality of life, or any combination of such effects.

When administration of two or more drugs occurs on the same day, the hypoxia-activated prodrug may be administered at least 30 minutes to about at least 2 hours before the taxane or the nucleoside chemotherapeutic is administered. The nucleoside chemotherapeutic may be administered after the hypoxia activated prodrug and after the taxane. The hypoxia-activated prodrug, the taxane, and the optional nucleoside analog chemotherapeutic may he administered in a plurality of cycles. By way of illustration, each cycle may comprise consecutively administering one or more of said drugs one after another on the same day, once a week for three consecutive weeks, followed by one week in which none of said drugs is administered.

In one embodiment, the hypoxia activated prodrug is T′H-302, which is administered at a dose ranging from 170 mg/m² to 340 mg/m² as an intravenous infusion over 30 minutes on days 1, 8, and 15 of a 28-day cycle; the taxane is nab-paclitaxel, which is administered at a dose ranging from 100 mg/m² to 125 mg/m² as an intravenous infusion over 30 minutes on days 1, 8, and 15 of every 28-day cycle; and the nucleoside analog is gemcitabine, which is administered at a dose ranging from 800 mg/m² to 1000 mg/m² on days 1, 8, and 15 of every 28-day cycle. Treatment cycles may be continued until cure or evidence of progressive disease or intolerable toxicity. In certain embodiments, the taxane is nab-paclitaxel, and/or the nucleoside analog is gemcitabine. These drugs are administered at a dose and a schedule that may be guided by doses and schedules approved by the U.S. Food and Drug Administration (FDA) or other regulatory body, subject to empirical optimization as part of a two- or three-drug combination according to this invention.

Exemplary conditions to which this invention may be applied include pancreatic cancer, particularly pancreatic ductal adenocarcinoma (PDAC). The drug combinations of this invention may cause a median increase in life expectancy in human patients of at least 30 or more days, at least 60 or more days, or 120 days or longer, compared with human patients having substantially the same condition but not treated with a combination of the invention.

Other aspects of the invention will be apparent from the description that follows.

### BRIEF DESCRIPTION OF THE DRAWINGS

FIG. 1 shows the tumor growth curves and Kaplan-Meier plots in the Hs766t, MIA PaCa-2, PANC-1 and BxPC-3 human PDAC xenograft models treated with different drug treatment regimens as described in Example 1, below. Briefly, different animal groups were treated with vehicle control (V); TH-302 (T) monotherapy; gemcitabine (G) and nab-paclitaxel (nP) combination therapy; or gemcitabine, nab-paclitaxel, and TH-302 combination therapy.

FIG. 2 shows the results of the hematological testing in the PANC-1 tumor-bearing nude mice and in CD-1 immunocompetent mice described in Example 1.

FIG. 3 shows the results of the blood chemistry testing for liver function in CD-1 immunocompetent mice described in Example 1.

FIG. 4 shows the results of the change of a panel of pharmacodynamic biomarkers by histology, immunohistochemistry staining or in situ assay, in the PANC-1 tumor-bearing nude mice described in Example 1.

FIG. 5 shows the von Frey neuropathy assay results in CD-1 immunocompetent Truce described in Example 1.

### DETAILED DESCRIPTION

Drug combinations or cocktails are sometimes used in treating cancer. However, the effect of two or more drugs in combination may not be particularly better than monotherapy, i.e., the use of one or the other drug by itself. Drugs may interact in a manner that decreases efficacy, increases unwanted side effects, or is otherwise not therapeutic for the patient.

This invention is based on the discovery that a hypoxia-activated prodrug such as TH-302 and a taxane such as paclitaxel and nab-paclitaxel, and optionally a nucleoside chemotherapeutic such as gemcitabine work especially well together in treating malignant conditions such as pancreatic cancer. The two and three drug combinations of this invention substantially inhibit tumor growth and increase survival in animal models of cancer and are expected to have similar benefit in human therapy. The benefit provided by the drug combinations of the invention will be, for many patients, more than that provided by any of the drugs alone or the combination of nab-paclitaxel and gemcitabine and beyond what could be predicted. Gemcitabine has been the standard of care for treating pancreatic cancer for many years; recently, a Phase 3 trial showed that gemcitabine and nab-paclitaxel combination therapy prolonged overall survival compared with gemcitabine monotherapy (median of 8.7 vs. 6.6 months), with treatment-related adverse events primarily relating to neutropenia and neuropathy. The FDA recently approved gemcitabine in combination with nab-paclitaxel for metastatic PDAC treatment. The present invention represents a significant advance in the treatment of this deadly disease.

In some patients, administration of a hypoxia-activated prodrug in combination with a taxane may be so effective that adding a nucleoside chemotherapeutic is unnecessary. This two-drug combination provided by the invention may also be more tolerable to some patients than the three-drug combinations provided by the invention.

The use of the drug combinations described herein represents an important advance in cancer management and treatment.

### I. Hypoxia Activated Prodrugs

Suitable for use in this invention is any hypoxia-activated prodrug that is inert or has less activity than the active form but that converts to the active form in vivo at or around a tumor site that is hypoxic, relative to normal tissues with physiological oxygenation. These drugs typically contain one or more bioreducible groups. The preparation and use of model hypoxia-activated prodrugs is described in WO 04/087075, WO 00/064864, WO 07/002931, and WO 08/083101, and US 2005/0256191, US 2007/0032455, and US 2009/0136521, each of which is incorporated herein by reference.

This invention may be conducted with hypoxia-activated prodrugs in the same class as bromo-isophosphoramide mustard (Br-IPM), having DNA bis-alkylator activity. Such compounds may have the structure shown in Formula I:

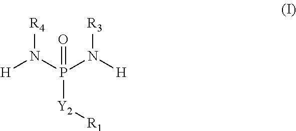

wherein Y₂ is O, S, NR₆, NCOR₆, or NSO₂R₆ wherein R₆ is C₁-C₆ alkyl, C₁-C₆ heteroalkyl, aryl, or heteroaryl; R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl; R₁ has the formula L-Z₃; L is C(Z₁)₂; each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl; or L is:

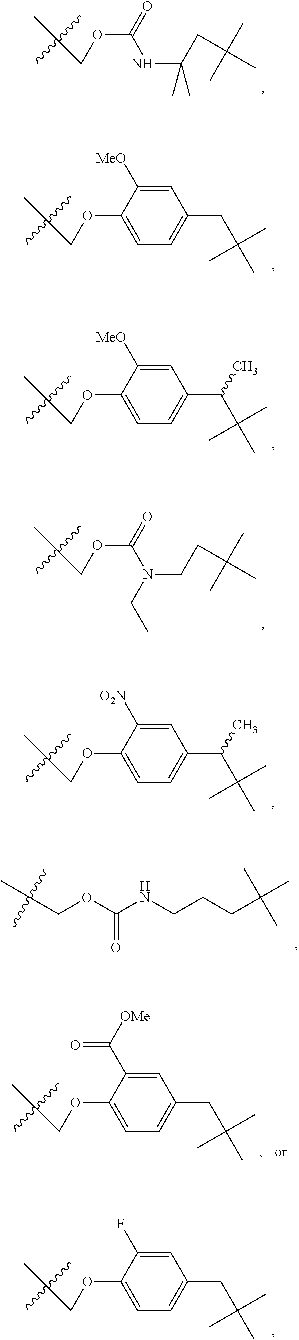

Z₃ is a bioreductive group having a formula selected from the group consisting of:

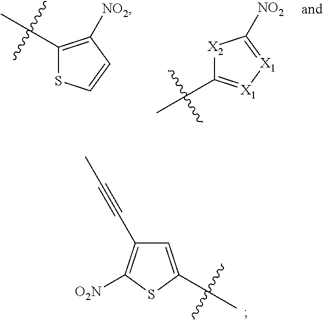

wherein each X₁ is independently N or CR₈; X₂ is NR₇, S, or O; each R₇ is independently C₁-C₆ alkyl, C₁-C₆ heteroalkyl, C₃-C₈ cycloalkyl, heterocyclyl, aryl or heteroaryl; and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl; or a pharmaceutically acceptable salt thereof.

Exemplary are TH-302 and TH-281, which respectively have the following structures:

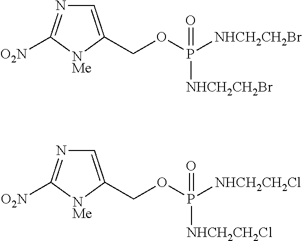

TH-302 and TH-281 convert to a cytotoxic agent selectively under hypoxic conditions in vivo at or around hypoxic tumor sites.

When used herein, reference to the compounds TH-302 and TH-281 are for illustrative purposes for the general class of compounds having the structure shown in Formula I. Unless expressly limited to a particular compound, the various aspects of the invention discussed in reference to TH-302 or TH-281 may be put into practice using TH-302 or TH-281 interchangeably, or using other hypoxia-activated prodrugs having the structure of Formula I, at the user's discretion.

In various embodiments, however, the hypoxia-activated prodrug is TH-302, which is administered in a daily dose of about 170 mg/m² to about 670 mg/m². Suitable administration schedules for doses of TH-302 in this range include the following:
once every three weeks at 670 mg/m²;
days one and eight of a twenty-one day cycle at 170, 240, 300, 340, 400, or 480 mg/m²;
days one, eight, and fifteen of a twenty-one day cycle (i.e., once a week, weekly) at 240, 340, 480, or 575 mg/m²;
days one, four, eight, and eleven of a twenty-one day cycle at 240 to 480 mg/m²;
days one to five of a twenty-one day cycle at 460 mg/m²;
days one, eight, and fifteen of a twenty-eight day cycle at 170 to 340 mg/m² or 240 to 575 mg/m²; and
days eight, fifteen, and twenty-two of a twenty-eight day cycle at 240 to 575 mg/m², e.g. 480 mg/m², once every two weeks at 240 to 670 mg/m².

Each of the above schedules can be considered a “cycle” of therapy. Patients will generally receive more than one cycle of therapy, although there may breaks of at least a day, and more generally a week or longer, between each cycle of therapy. Other compounds of Formula I are generally dosed in accordance with the above schedules and amounts, with the amount adjusted to reflect how active the compound is relative to TH-302.

### II. Taxanes

Taxanes are a family of compounds that comprises diterpenes produced by the plants of the genus Taxus (yews), and chemically synthesized equivalents and analogs. Examples in current clinical use include paclitaxel (Taxol®) and docetaxel (Taxotere®). The principal mechanism of action of the taxane class of drugs is thought to be disruption of microtubule function by stabilizing GDP-bound tubulin in the microtubule, thereby acting as a spindle poison to inhibit mitosis.

Taxanes that can be used in this invention include 9-dihydrotaxol analogs in general having a chemical structure according to Formula II.

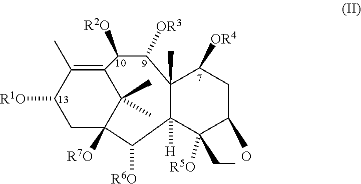

R₂, R₄, R₅ and R₇ in formula (II) are independently hydrogen, alkyl, alkanoyl or aminoalkanoyl. R₃ in formula (II) is hydrogen, alkyl or aminoalkanoyl. R₆ in formula (II) is hydrogen, alkyl, alkanoyl, aminoalkanoyl or phenylcarbonyl (—C(O)-phenyl), R₁ has the following structure:

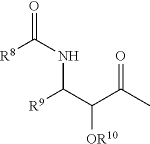

in which R₈ is hydrogen, alkyl, phenyl, substituted phenyl, alkoxy, substituted alkoxy, amino, substituted amino, phenoxy or substituted phenoxy; R₉ is hydrogen, alkyl, hydroxyalkyl, alkoxyalkyl, aminoalkyl, phenyl or substituted phenyl; and R₁₀ is hydrogen, alkanoyl, substituted alkanoyl or aminoalkanoyl. See U.S. Pat. No. 5,352,806, incorporated herein by reference.

Exemplary taxane include paclitaxel, which has the following structure.

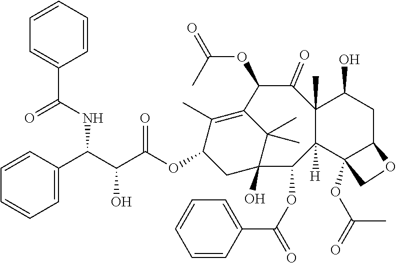 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Paclitaxel has been used to treat patients with lung, ovarian, breast, head and neck career, and advanced forms of Kaposi's sarcoma, and to prevent restenosis.

To facilitate administration, improve stability, and/or reduce unwanted toxicity, compounds of the taxane class can be bound to or microencapsulated within a cross-linked polymer as described in U.S. Pat. No. 5,439,686, incorporated herein by reference. The active pharmacological agent can be substantially all contained in a polymeric shell (preferably having a largest cross-sectional dimension of no greater than about 10 microns). The shell comprises a biocompatible polymer that is substantially cross-linked by way of disulfide bonds. The pharmacologically active agent is suspended in the shell in a biocompatible aqueous liquid. A suitable biocompatible polymer for this purpose is a human or otherwise non-immunogenic protein with a molecular weight of between about 10 and 100 kDa, as exemplified by albumin.

The US. Food and Drag Administration (FDA) has approved paclitaxel albumin-bound particles (referred to as nab-paclitaxel and commercially available under the trade name Abraxane®) for use in treating recurrent or metastasized breast cancer. Nab-paclitaxel is also approved for treating locally advanced or metastatic non-small cell lung, cancer (NSCLC) patients who are not candidates for curative surgery or radiation therapy. Nab-paclitaxel is also approved in combination with gemcitabine for treating metastatic pancreatic ductal adenocarcinoma.

When used in the general description of this invention, reference to the compounds “paclitaxel” or “'nab-paclitaxel” are for illustrative purposes for the general class of compounds having the structure shown in Formula II, optionally bound to or encapsulated in a biopolymer or protein such as albumin. Unless expressly limited to a particular compound, the various aspects of the invention discussed in reference to paclitaxel or nab-paclitaxel may be put into practice using drugs having the structure of Formula II, at the user's discretion. While any taxane can be used at any FDA approved dose in accordance with the methods of the invention, in various embodiments, the taxane is Nab-paclitaxel, which is administered at a dose ranging from 100 mg/m² to 125 mg/m² as an intravenous infusion over 30 minutes on days 1, 8, and 15 of every 28-day cycle

### III. Nucleoside Analogs and Other Chemotherapeutics

Therapeutic agents that can be used in combination with a hypoxia-activated prodrug and a taxane in accordance with the invention include nucleoside analogs. Nucleoside analogs are designed to interfere with DNA replication. For example, some nucleoside analogs are incorporated into DNA synthesis during the process of mitosis, but thereafter prevent lengthening of the replicating DNA. They may also irreversibly bind and inhibit ribonucleotide reductase (RNR), thus preventing or decreasing synthesis of deoxynucleotides.

Included in the nucleoside analog class of drugs are chemotherapeutics having the structure shown in Formula III:

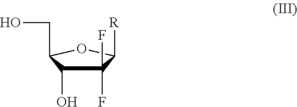

where R is a nitrogen-containing monocyclic or heterocyclic aromatic compound chosen so that the compound may mimic a nucleoside and block enzymatic reactions associated with DNA biosynthesis, R may be selected from the following:

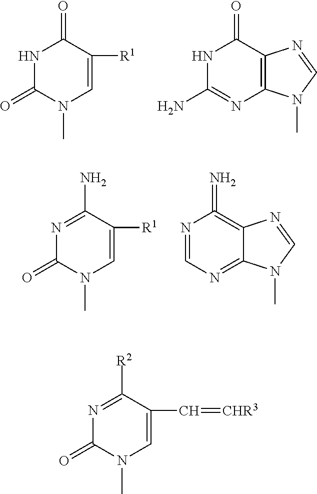

where R₁ is hydrogen, methyl, bromo, fluoro, chloro or iodo; and R₂ is hydroxy; R₃ is hydrogen, bromo, chloro or iodo. See U.S. Pat. No. 4,808,614, incorporated herein by reference.

An exemplary nucleoside analog suitable for use in accordance with the invention is the compound gemcitabine (distributed under the trade name Gemzar®), which has the following structure:

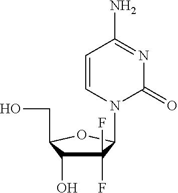

Gemcitabine is currently in use for treating various carcinomas, specifically non-small cell lung cancer, pancreatic cancer, bladder cancer and breast cancer, and is under investigation for use in esophageal cancer and lymphomas.

When used in the general description of this invention, reference to the compound gemcitabine is for illustrative purposes for the general class of compounds having the structure shown in Formula III. Unless expressly limited to a particular compound, the various aspects of the invention discussed in reference to gemcitabine may be put into practice using gemcitabine or other nucleoside analogs with chemotherapeutic activity having the structure of Formula III, at the user's discretion. In various embodiments of the methods of the invention, however, the compound gemcitabine is administered at a dose ranging from 800 mg/m² to 1000 mg/m² on days 1, 8, and 15 of every 28-day cycle.

Alternatively, or in addition, the therapeutic combinations used as medicaments for treating cancer according to this invention may comprise other known chemotherapeutic agents and/or radiation, chosen with reference to previous experience with such agents in the treatment of cancers of the particular tissue type and stage that has been diagnosed in the patient being treated.

### IV. Formulation

This invention encompasses the use of a hypoxia-activated prodrug, exemplified by compounds of Formula I, such as TH-302; a taxane as exemplified by compounds of Formula II, such as protein-bound paclitaxel, and optionally a nucleoside chemotherapeutic as exemplified by compounds of Formula III, in the manufacture of a single medicament (which contains two or more active drugs) or medicament combination that may be manufactured, distributed or used for therapy as described below. A “medicament combination” as used herein refers to two or more medications that are used in combination and may be co-formulated (admixed together) or separately formulated (not admixed or otherwise combined together in a single unit dose form).

Formulations of TH-302 or TH-281 suitable for parenteral or intravenous injection and methods for administering them in the treatment of cancer that are suitable for use in practice of the present invention are described in WO 07/002931, WO 08/083101, WO 10/048330, WO 12/142520, and WO 13/126539.

While the various methods of the invention are illustrated specifically with TH-302, nab-paclitaxel, and gemcitabine, other particular compounds, formulations, and dosing schedules of the invention can be assessed for safety and efficacy in preclinical models and clinical trials. In such studies, one combines a compound, formulation, or drug combination of the invention into an in vitro culture of an established cell line corresponding to the target cancer or a preclinical animal model, for example, a homograft or allograft model using tumor cell lines derived from the same species, or a xenograft of human tumor cells in an immune-compromised animal. Using such systems and models, the investigator may determine, for example, the maximum tolerable dose and the dose required for a significant beneficial therapeutic effect using such models.

Depending on efficacy and side effect profile, a hypoxia-activated prodrug, a taxane, and optionally a nucleoside chemotherapeutic may be distributed and administered separately in a treatment of a particular disease or condition. Alternatives are as follows: a hypoxia-activated prodrug may be combined with a taxane administration together, optionally with a nucleoside analog in a separate formulation; or a taxane and a nucleoside analog may be combined for administration together, and administered with a hypoxia-activated prodrug in a separate formulation; or a hypoxia activated prodrug and a nucleoside analog may be combined, and administered with a taxane in a separate formulation; or a hypoxia-activated prodrug, a taxane, and a nucleoside analog may be combined in a single formulation; or the drugs may be separately formulated and administered.

The invention also encompasses various combinations of agents for marketing or distribution together. Such combinations are optionally marketed and distributed in kit form. The combinations or kits may comprise separate packs of an effective amount of a hypoxia-activated prodrug, exemplified by Formula I, such as TH-302; a taxane as exemplified in Formula II, such as protein-encapsulated paclitaxel or nab-paclitaxel, and optionally a nucleoside chemotherapeutic as exemplified in Formula III, such as gemcitabine. The combination or kit will be suitably packaged and may also contain or be marketed in combination with written instructions that direct the clinician on the use of the combination or elements of the kit for chemotherapy in accordance with the invention.

### V. Use in Treatment

This invention encompasses the commercial and clinical use of a hypoxia-activated prodrug, exemplified by Formula I, such as TH-302; a taxane as exemplified in Formula II, such as protein-encapsulated paclitaxel or nab-paclitaxel, and optionally a nucleoside chemotherapeutic as exemplified in Formula III, such as gemcitabine. Such combinations are used in the prophylactic or therapeutic treatment of a condition or disease, such as cancer, that is caused, mediated, or propagated by undesired cell growth, hyperproliferation, malignancy, or tumor formation.

The drug combinations of this invention can be used therapeutically in cancers of various types, especially solid tumors comprising or expected to develop hypoxic regions. Examples include but are not limited to cancer of the adrenal gland, bone, brain, breast, bronchi, colon and/or rectum, gallbladder, head and neck, kidneys, larynx, liver, lung, neural tissue, pancreas, prostate, parathyroid, skin, stomach, and thyroid. Other examples include acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy tell tumor, intestinal ganglioneuroma, hyperplastic corneal nerve tumor, islet cell carcinoma, Kaposi's sarcoma, leiomyoma, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteosarcoma, osteogenic and other sarcoma, ovarian tumor, pheochromocytoma, polycythermia vera, glioma, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, veticulum cell sarcoma, and Wilm's tumor,

The hypoxia-activated prodrug, the taxane, and the nucleoside chemotherapeutic may be administered simultaneously or sequentially in any effective combination at the election of the managing clinician, upon consideration of previous experience, and the condition and history of the patient. In various embodiments in which the three drugs are coadministered or administered on the same day, the hypoxia activated prodrug will be administered first, with at least a half hour (up to 2 or even 4 hours) delay from the completion of administration of the hypoxia activated prodrug until the administration of the second drug.

### VI. Other Treatments

The treatment methods of the invention may result in side effects, which may be treated in accordance with other treatments of the invention.

Intertriginous rash may be prevented or treated by application of Preparation H to the perineal area, around the anus, under the arms, and other areas where there are skin folds. Prophylactic treatment may begin prior to TH-302 administration (15 minutes prior to infusion). A cool pack may be applied to the inguinal region during TH-302 infusion After the infusion and after bowel movements, desitin cream (maximum strength) may be applied to the perineal area. Silvadene 1% cream and triamcinclone 0.1% cream can be applied to affected areas. In severe cases, discontinue treatment until the rash clears. Anal mucositis can be prevented or treated with the same treatments; however, cryotherapy during infusion and pain control (NSAIDS, CGRP inhibitors, or narcotics) may also be required for anal mucositis.

Hand-foot skin reaction can be prevented with ammonium lactate 12% cream (Amlactin®) or heavy moisturizer (Vaseline) twice daily or with cryotherapy. Hand-foot skin reaction can be treated with ammonium lactate 12% cream twice daily and clobetasol 0.05% cream once daily. Pain control may also be required and obtained using NSAIDS, CGRP inhibitors, or narcotics.

Oral mucositis can be prevented or treated with oral cryotherapy during infusion. Treatment can be achieved with elixir (nydrocortisone 200 mg, Nystatin 2 million units, tetracycline 1500 mg, Benadryl equal to 250 cc) by swish and swallow 1 tsp t.i.d. and pain control (as above).

Injection site reaction can be treated with extremity elevation and daily warm compresses. Severe cases may require treatment as with a wound, plastic surgery, and oral antibiotics.

Hyperpigmentation can be prevented with sunscreen (SPF 30) to all exposed skin, Treatment can be achieved with ammonium lactate 12% cream (and continued use of sunscreen) or, for more severe cases, hydroquinone 4% cream.

### VII. Definitions

Reference to any drug or active agent in this disclosure includes any and all isomers, stereoisomers, pharmaceutically compatible salts, solvates, and pharmaceutical compositions thereof that retain at least some of the physiological or chemotherapeutic effects of the drug itself, unless such isomers, salts, solvates, and/or compositions are explicitly excluded. Any such compound may be used as an alternative to the drug itself to improve efficacy, tolerability, delivery, or pharmacokinetics, or simply by choice within the good judgment of the manufacturer, distributor, pharmacist, clinician, or end user

The therapeutic agents referred to as “TH-302” and “TH-281” are exemplary hypoxia-activated prodrugs, which are described in more detail above. The therapeutic agent referred to as “nab-paclitaxel” is paclitaxel bound to albumin particles, commercially available under the trade name Abraxane®, marketed by Celgene.

An “active agent” or “pharmaceutical” is a compound with a desired pharmacological effect. It includes all pharmaceutically acceptable forms of the active agent described. Unless explicitly stated otherwise, all embodiments of the invention may be practiced with any one or more different isomers, stereoisomers, and pharmaceutical salts of each of the active ingredients that has the desired effect.

A “chemotherapeutic agent” is a pharmaceutical compound that is given to a cancer patient primarily to eradicate, diminish, stabilize, or decrease the growth rate or metabolism of one or more malignant tumors in the patient. Included are nucleoside analogs such as gemcitabine. The more general term “therapeutic agent” includes chemotherapeutics and radiation therapy.

A “prodrug” is a compound that, after administration, is metabolized or otherwise converted to a biologically active or more active agent with respect to at least one beneficial property or effect.

A “hypoxia-activated prodrug” is a prodrug that is less active or inactive, relative to the active form of the drug, to which it is activated in vivo. It contains one or more bioreducible groups. The term includes prodrugs that are activated by reducing agents and enzymes, including single electron transferring enzymes (such as NADPH cytochrome P450 reductases) and two electron transferring (or hydride transferring) enzymes. Exemplary are 2-nitroimidazole triggered hypoxia-activated prodrugs.

The terms “patient” and “subject” are used in this disclosure to refer to a mammal being treated or in need of treatment for a condition such as cancer. The terms include human patients and volunteers, non-human mammals such as a non-human primates, large animal models and rodents.

“Administering” or “administration of” a drug to a patient refers to direct administration, which may be administration to a patient by a medical professional or may be self-administration, and/or indirect administration, which may be the act of prescribing a drug. For example, a physician or clinic that instructs a patient to self-administer a drug or provides a patient with a prescription for a drug is administering the drug to the patient.

The terms “dose” and “dosage” refer to a specific amount of active or therapeutic agent(s) for administration at one time. A “dosage form” is a physically discrete unit that has been packaged or provided as unitary dosages for subjects being treated. It contains a predetermined quantity of active agent calculated to produce the desired onset, tolerability, and therapeutic effect.

A “therapeutically effective amount” of a drug refers to an amount of a drug that, when administered to a patient to treat a condition such as cancer, will have a beneficial effect, such as alleviation, amelioration, palliation or elimination of one or more symptoms, signs, or laboratory markers associated with the active or pathological form of the condition. Desirable effects for cancer patients may include reducing the rate of tumor growth, causing tumors to shrink, causing circulating markers of the cancer to decrease, and improving progression-free or overall survival.

### EXAMPLES

The following examples are intended for illustration only and should not be construed to limit the scope of the claimed invention. The principal agents used are TH-302, as a representative species of hypoxia-activated drugs; nab-paclitaxel as a representative species of taxane, and gemcitabine as a representative species of nucleoside analog chemotherapeutics.

### Example 1

### Animal Model Demonstration of Safely and Efficacy

Dosing for the animal studies described below were selected as follows. In some current human clinical trials, TH-302 is given on once-a-week schedules (QW) of three weeks on, one week off (at a dose of 240 or 340 mg/m²). When TH-302 and gemcitabine are co-administered on the same day, the combination is generally more effective if the TH-302 is given 2 to 4 hours before the gemcitabine (Liu et al., Cancer Chemother Pharmacol. 69:1487-1498, 2012). Nab-paclitaxel has been shown to increase the intratumor concentration of gemcitabine (Von Hoff et al., J Clin Oncol. 29:4548-4564, 2011). In the clinical Phase 3 trial (NCT00844649), nab-paclitaxel was administered followed by gemcitabine on once-a-week schedules (QW) of three weeks on, one week off. In xenograft models, gemcitabine is administered at 60 or 80 mg/kg in four three-day cycles (Q3D×4) (Teicher et al., Cancer Res. 6:1016, 2000; Marriman et at., invest New Drugs. 14:243, 1996). Therefore, a ‘same day’ administration of TH-302, nab-paclitaxel and gemcitabine with a Q3D×5 (every-three-days-for-five-times) regimen was selected. MTD (maximum tolerated dose) studies in xenograft nude mice models showed that a combination of 75 mg/kg TH-302, 30 mg/kg nab-paclitaxel, and 60 mg/kg gemcitabine Q3D×2 was toxic to some degree in ⅓ of the animals. However, 50 mg/kg TH-302, 30 mg/kg nab-paclitaxel, and 60 mg/kg gemcitabine was non-toxic and caused no more than 10% loss in body weight. Accordingly, the dosages used in the animal studies described herein were as follows: TH-302 at 50 by intraperitoneal injection (i.p.); nab-paclitaxel at 30 mg/kg by intravenous injection (i.v.); and gemcitabine at 60 mg/kg i.p., nab-paclitaxel was administered 2 hours after TH-302 administration, and nab-paclitaxel administration was followed one hour later by gemcitabine administration. The therapy was administered every three days for five cycles (Q3D×5).

The animal studies were conducted using, xenograft models for pancreatic ductal adenocarcinoma (PDAC). Tumors were generated in the flanks of nude mice by subcutaneous injection of established human PDAC cell lines. Four PDAC xenograft models were established by subcutaneous implantation of either Hs766t, MIA PaCa-2, PANC-1 or BxPC-3 cells into the flank of nude mice. Mice were implanted subcutaneously in the right flank with 5×10⁶ cells in 30% Matrilgel™ at a dose volume of 200 μL. Mice bearing similar tumor size were randomly assigned into different groups when tumor size reached 150 mm³. A ‘same day’ administration of TH-302 (T), nab-paclitaxel (nP), and gemcitabine (G) with a Q3D×5 regimen was employed: T, 50 mg/kg, i.p.; nP: 30 mg/kg, i.v.; and G: 60 mg/kg i.p., Saline treated animals served as vehicle control (V). Antitumor activity of the G+nP+T triplet combination therapy was assessed, and compared with the G+nP doublet combination therapy.

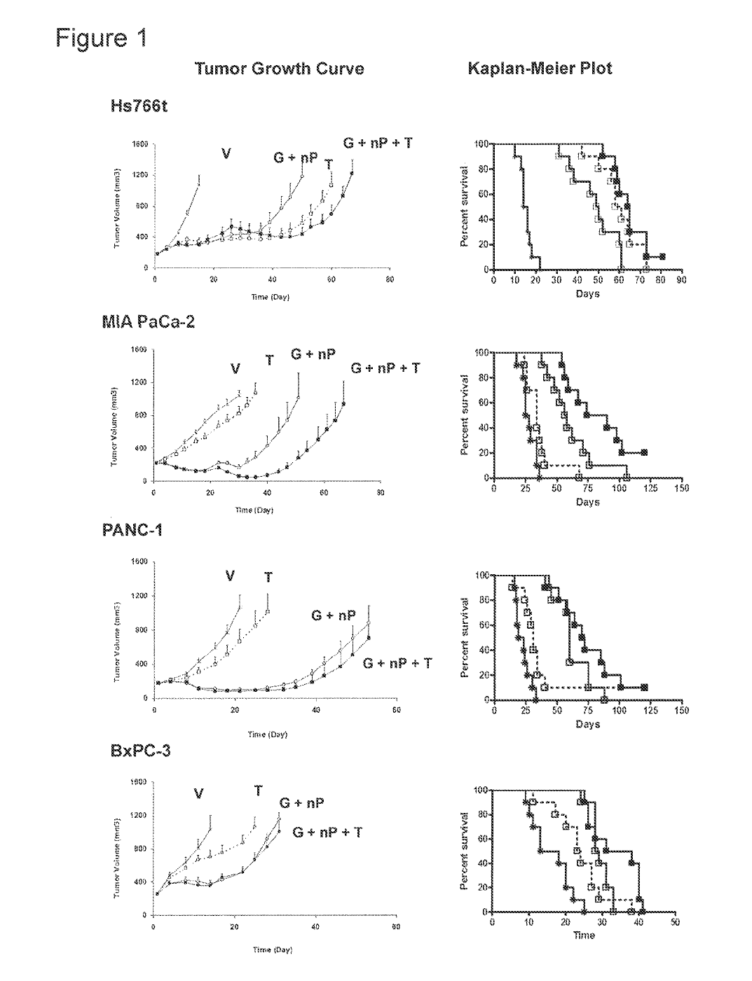

FIG. 1 shows the results (mean±SEM). As demonstrated by tumor growth inhibition (TGI) and Kaplan-Meier analysis of median time (MT) to tumor size of 1000 mm³to evaluate antitumor activity, the G+nP+T combination therapy exhibited enhanced efficacy compared with T monotherapy or the G+nP combination therapy in all four models. In the Hs766t xenograft model, combining nP with G statistically inhibited tumor growth compared with V, with a TGI of 85.2%. T monotherapy yielded a TGI of 82.1%. The TGI of T+nP+G triplet combination therapy was 86.1%. Tumor growth delay 1000 mm³ (TGD1000) was determined as the difference in time required for the mean tumor size to reach 1000 mm³ between treated group and vehicle control group. TGD1000 of T monotherapy and G+nP doublet combination therapy groups was 47 and 35 days, respectively, while TGD1000 of G+nP+T triplet combination therapy group was 53 days. A Kaplan-Meier plot was used to calculate MT to reach the size of 1000 mm³, the triplet combination therapy treated group showed MT of 64.5 days, which was significantly prolonged compared with T monotherapy of 59.5 days or G+nP doublet combination therapy of 49.5 days. (see Table 1, below). In the MIA PaCa-2, PANC-1 and BxPC-3 xenograft models, G+nP doublet combination therapy showed superior antitumor activity, and when T was added, the efficacy was enhanced. In all 4 models tested, triplet combination therapy statistically increased the antitumor activity and prolonged the survival compared with T monotherapy; In 3 of 4 models, triplet combination therapy statistically increased the antitumor activity and prolonged the survival compared the doublet combination therapy, reflected on tumor growth inhibition or median time to grow to 1000 mm³.

In three of the four models, the triplet combination therapy increased the complete response rate (CR) relative to the G+nP combination therapy or the T monotherapy. CR was defined as disappearance of tumor or tumor size is <100 mm³ at a study point. Of note, the CR rate of the G+nP+T combination therapy in the PANC-1 model reached 100%, compared to 0% and 60% in the T monotherapy and G+nP combination therapy groups, respectively. In the MIA PaCa-2 model, CR rate in the triplet group was 90%, compared to 0 and 10% in the T monotherapy and G+nP doublet combination therapy groups, respectively.

Another series of studies were conducted to investigate the treatment-induced body weight (BW) loss in both PDAC tumor bearing animals and CD-1 immunocompetent mice. Body weight was measured daily or twice a week. As shown in Table 2, below, maximal BW loss (percentage of mean maximal BW loss compared with pretreatment) in the T monotherapy group was 0. G+nP combination therapy induced maximal BW loss from 4.2 to 15.8% in 7 of 8 studies in which G+nP+T combination therapy did not induce more body weight loss. One study conducted in PANC-1 tumor bearing mice showed that the triplet combination therapy induced 8.5% BW loss compared with no loss in the doublet group (PANC-1 #1). This study was repeated as shown in PANC-1 #3, the doublet treatment induced 11.1% BW loss while the triplet treatment induced 12.3% BW loss. Overall, in 2 of 3 studies conducted in PANC-1 xenograft models, triplet treatment did not add body weight loss on the doublet group. Using BW loss as a read-out, there was no additive toxicity from the triplet combination therapy compared with the doublet combination therapy.

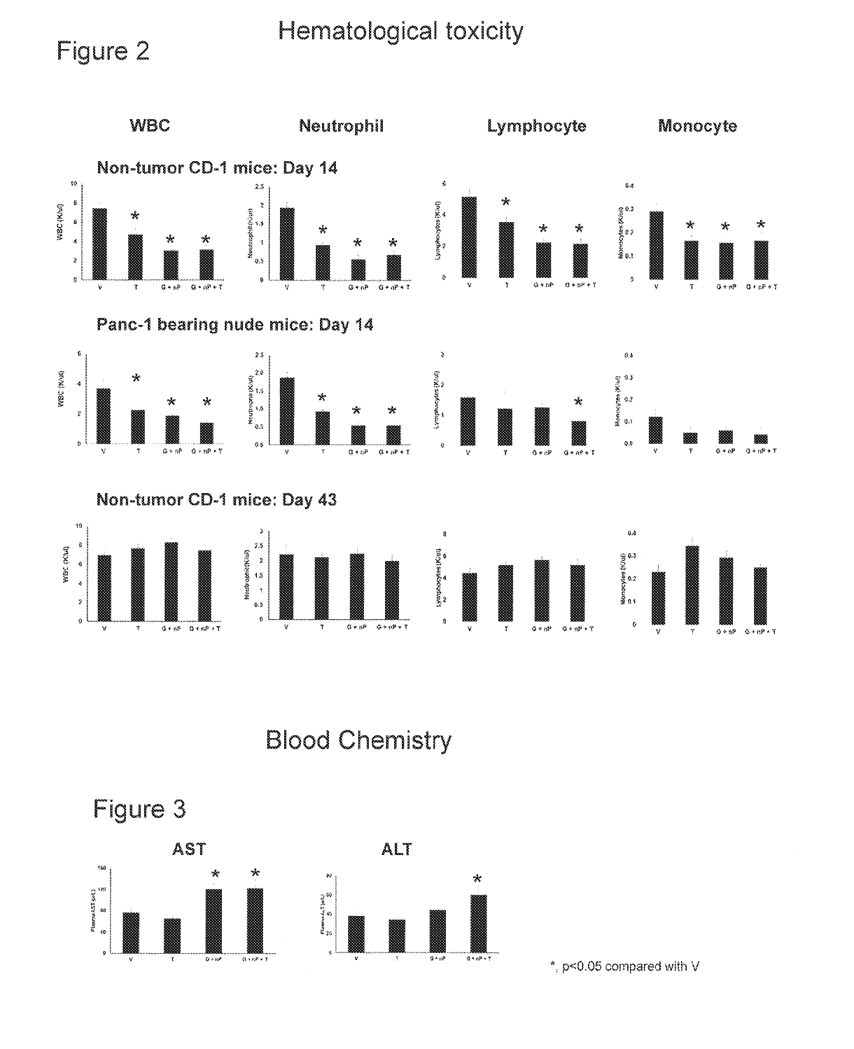

To determine the effect of combination therapy on hematopoiesis, the same treatment was employed in PANC-1 tumor bearing nude mice as well as immunocompetent CD-1 mice. Twenty-four hrs. after the last treatment, the animals were euthanized under CO₂, blood was collected via heart puncture, and hematological analysis was performed using a Hemavet 950™ blood analyzer. As shown in FIG. 2 (mean±SEM), G+nP combination therapy or T monotherapy reduced neutrophils, lymphocytes, and monocytes, and complete white blood cell count (WBC), compared with V(*p<0.05 compared with V). There was no further decrease in neutrophil, lymphocyte, or monocyte count when T was added to G+nP combination therapy compared to G+nP combination therapy. The G+nP+T combination therapy did not add hematoxicity compared with the G+nP combination therapy. Another study conducted in CD-1 mice showed that 43 days after the last treatment, all the white blood cell parameters were recovered to normal levels.

To determine the effect of combination treatment on blood chemistry, immunocompetent CD-1 mice were treated with V, T monotherapy, G+nP doublet combination therapy or G+nP+T triplet combination therapy. Twenty four hrs, after the last treatment, the animals were euthanized under CO₂, blood was collected via heart puncture, and plasma was obtained for the biochemistry assay. As shown in FIG. 3 (mean±SEM), plasma AST levels were elevated after G+nP combination therapy, but G+nP+T combination therapy did not further increase the levels of these liver function indicators. There was a significant elevation of ALT level in the G+nP+T combination therapy group compared with V; however, there was no statistical difference between G+nP+T combination therapy vs. G+nP combination therapy treated animals. The G+nP+T combination therapy did not add hepatotoxicity compared with the G+nP doublet combination therapy.

We used a panel of biomakers to characterize Hs766t, MIA PaCa-2, PANC-1 and BxPC3 xenograft tumors. The four tumor types showed different hypoxia levels, vasculature densities, EMT (epithelial-mesenchymal transition) status, and stroma components. We chose a moderate hypoxic level tumor model, PANC-1, to investigate the pharmacodynamic changes caused by the different treatments, PANC-1 tumor-bearing animals were treated with vehicle, TH-302, G+nP, and G+nP+T when tumor size was approximately 500 mm³. One day after the last treatment, tumors were harvested, fixed, paraffin-embedded, and sectioned for staining with a panel of histology and immunohistochemistry markers.

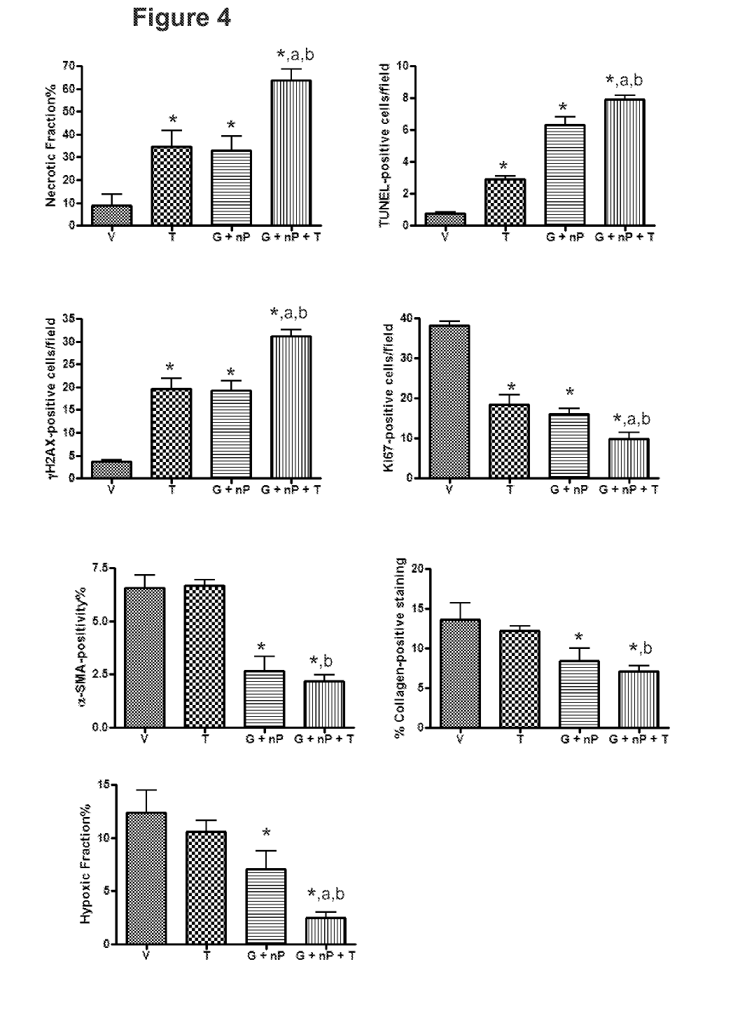

As shown in FIG. 4, tumor necrotic fraction evaluated by Masson's Trichrome histology staining was significantly increased to 64% after the G+nP+T triplet combination, compared 9% in V, and 35% in T alone or 33% in the G+nP doublet group (p<0.01). Apoptotic cells, detected by TUNEL assay, were observed in both cancer cells or in the stromal compartment in the non-necrotic regions, G+nP+T triplet therapy significantly increased the number of apoptotic cells (8±0.3 per field, compared to 0.8±0.1, 3±0.2, 6.3±0.5, in V, T alone and G+nP, respectively, p<0.05). γH2AX foci formation was employed to assess treatment-induced DNA damage. Similar to as observed for apoptosis, γH2AX-positive cells were increased in all three drug treated groups compared with V. 31±1.3 positive cells/field in the G+nP+T triplet group, compared with 19 in T alone or G+nP and less than 4 in V (p<0.05). Cells with DNA damage were evenly distributed in both the hypoxic and oxic compartments in non-necrotic regions. Ki67 is a marker of cell proliferation, labeling all active phases of the cell cycle including S, G2, and mitosis. Immunostaining demonstrated that Ki67-positive cells were significantly reduced after the drug treatments but to a greater magnitude in the G+nP+T triplet combination group (FIG. 4).

To determine the effect of combination therapy on tumor microenvironment, tumor stroma and hypoxia were investigated. Tumor stroma is composed of extracellular matrix protein and cellular elements. Thus, collagen I and III, as main parts of extracellular matrix were evaluated by Picrosirius red staining. Activated fibroblasts in the stroma were analyzed by α-SMA. By morphometric analysis, G+nP significantly reduced extracellular collagen and α-SMA expression, and no further reduction was observed in the G+nP+T group. T-alone had no effect on α-SMA or collagen expression compared to V. Of note, by picrosirius red staining, after treatment of G+nP or G+nP+T, the thinner, disrupted and disorganized collagen was appeared compared with V. T alone did not reduce the hypoxic fraction (HF, 10.6±1.1% vs. 12.4+2% in V) however, by adding T, the triplet significantly reduced the hypoxic level, with HF of 2.5±0.5%, compared with 7.1±1.7% in G+nP group (p<0.05). There was no co-localization between exogenous pimonidazole- and endogenous CAIX-positive cells, CA-IX was found in the necrotic cells as well. These results indicate that CA-IX is not a biomarker of hypoxia in this PDAC model.

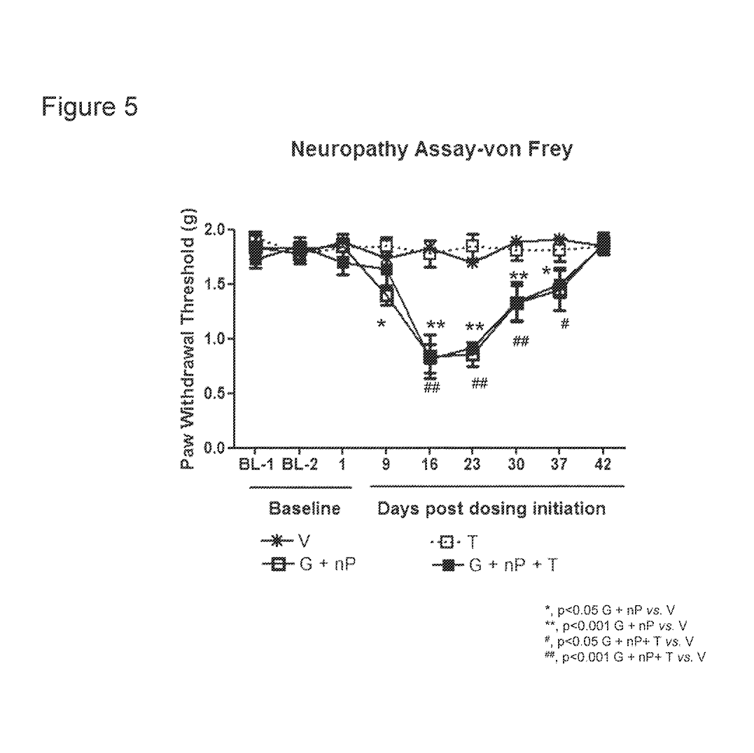

To determine the effect of combination therapy on the nervous system, male CD-1 mice were randomly divided into 4 groups with 10 mice each. Mechanical hyperalgesia tests were performed prior to the initial treatment and during the study by a trained observer who was unaware of the animal group identities. As shown in FIG. 5, the von Frey neuropathy assay demonstrated that, at baseline, there were no differences in response to mechanical stimulus between mice in the four groups (p>0.05, two-way ANOVA). However, at 9 days after the initiation of treatment, G+nP combination therapy treated mice exhibited a significant hind-paw mechanical hyperalgesia compared to the V controls (p<0.05, two-way ANOVA). This hyperalgesia persisted to 37 days with peaks at day 16 and day 23 (p<0.001, two-way ANOVA), and gradually returned to baseline at day 43. A similar change in mechanical hypersensitivity was observed in mice treated with G+nP+T combination therapy compared to V over time. There was no difference between G+nP+T combination therapy treated and G+nP combination therapy treated animals. Of note, no mechanical hyperalgesia was detected in mice treated with T in various time points tested (p>0.05, two-way ANOVA). The results suggest that the triplet of G+nP+T combination therapy did not add neuropathy compared with G+nP combination therapy.

Certain results of these studies are presented in tabular form below.

TABLE 1
Summary of antitumor activity of TH-302 monotherapy, gemcitabine and
nab-paclitaxel doublet and gemcitabine, paclitaxel and TH-302 triplet
in the Hs766t, MIA PaCa-2, PANC-1 and BxPC-3 PDAC xenograft models.

| | | | | | TV 1000 mm³ | |
|---|---|---|---|---|---|---|
| | | | TGD₁₀₀₀ | CR | as Endpoint | |
| Model | Group | TGI | (vs. vehicle) | of 10 | MT (day) | ILS |
| Hs766t | Vehicle | | | 0 | 15 | |
| | TH-302 | 82.1%* | 47 | 0 | 59.5*^{,a} | 297% |
| | G + nP Doublet | 85.2%* | 35 | 0 | 49.5* | 230% |
| | G + nP + T Triplet | 86.1%* | 53 | 1 | 64.5*^{,a,b} | 330% |
| MIA | Vehicle | | | 0 | 27 | |
| PaCa-2 | TH-302 | 27% | 6 | 0 | 34 | 26% |
| | G + nP Doublet | 107%* | 23 | 1 | 57* | 111% |
| | G + nP + T Triplet | 120%*^{,a,b} | >39 | 9 | 82*^{,b} | 204% |
| PANC-1 | Vehicle | | | 0 | 21 | |
| | TH-302 | 44.5% | 7 | 0 | 31 | 48% |
| | G + nP Doublet | 107.4%* | 34 | 6 | 60* | 186% |
| | G + nP + T Triplet | 110.3%*^{,a,b} | >34 | 10 | 71*^{,b} | 238% |
| BxPC-3 | Vehicle | | | 0 | 16 | |
| | TH-302 | 43% | 11 | 0 | 24 | 52% |
| | G + nP Doublet | 84.7%* | 16 | 0 | 29* | 84% |
| | G + nP + T Triplet | 86.5%*^{,b} | 18 | 0 | 35*^{,b} | 123% |

TGI: Tumor Growth Inhibition
TGD₁: Tumor Growth Delay
CR: Complete Response
MT: Median Time to reaching the size of 1000 mm³
ILS: Increased Life Span
*p < 0.05 compared with V
^{a}p < 0.05 compared with G + nP doublet
^{b}p < 0.05 compared with T monotherapy

TABLE 2
Summary of TH-302 monotherapy, gemcitabine and nab-paclitaxel doublet
and gemcitabine, paclitaxel and TH-302 triplet induced body weight
loss in the PDAC tumor bearing and CD-1 non-tumor bearing mice.

| | | MIA | PANC-1 | PANC-1 | PANC-1 | | Non- | Non- |
|---|---|---|---|---|---|---|---|---|
| | Hs766t | PaCa-2 | #1* | #2* | #3* | BxPC3 | tumor | tumor |
| | Nude ♀ | Nude ♀ | Nude ♀ | Nude ♀ | Nude ♀ | Nude ♀ | CD-1 ♀ | CD-1 ♂ |
| | (n = 10) | (n = 10) | (n = 10) | (n = 5) | (n = 10) | (n = 10) | (n = 6) | (n = 10) |
| Vehicle | 0 | 0 | 0 | 0 | 0 | 0 | 0 | 0 |
| T | 0 | 0 | 0 | 0 | 0 | 0 | 0 | 0 |
| G + nP Doublet | 13% | 14.2% | 0 | 15.8% | 11.1% | 9% | 4.2% | 5.4% |
| G + nP + T Triplet | 12.3% | 13% | 8.5% | 16.8% | 12.3% | 8.2% | 3% | 1.7% |

*three different studies

### Example 2

### Demonstration of Efficacy in Humans

A human clinical trial can be conducted to demonstrate the safety and tolerability, define the maximum tolerated dose (MTD), and demonstrate the efficacy of the combination of TH-302 with gemcitabine and nab-paclitaxel. Suitable patients for such a trial include previously untreated subjects with locally advanced unresectable or metastatic pancreatic adenocarcinoma. The dose escalation part of the trial can be conducted in both subject populations (previously untreated subjects with locally advanced unresectable and metastatic pancreatic adenocarcinoma), and the cohort expansion part at the MTD can include only subjects with metastatic disease.

As shown in Example 1, TH-302 markedly enhanced tumor growth delay of nab-paclitaxel in combination with gemcitabine in human tumor pancreatic cancer xenografts in immunosuppressed mice. The weight loss observed in the mice when treated with nab-paclitaxel and gemcitabine was not increased by the addition of TH-302 in these models.

A human clinical trial can include analysis of any of a series of biomarkers. Enhanced sensitivity to TH-302 has been observed in cell lines with mutations in BRCA1 or BRCA2. The prevalence of BRCA1 and BRCA2 mutations in pancreatic cancer patients is between 5% and 19%, Thus, a trial can include collection of serum, plasma, and tissue samples for analysis of any markers useful for identifying suitable patient populations and/or liar monitoring the course of therapy. Such markers include hypoxia biomarkers, BRCA1 or BRCA2 genetic mutations, or functionally related markers that may identify subjects most likely to benefit from the TH-302., nab-paclitaxel, and gemcitabine regimen.

The starting dose of TH-302 can be 170 mg/m². TH-302, nab-paclitaxel, and gemcitabine have partly overlapping toxicities, especially myelosuppression. The protocol described here foresees to manage toxicities by using a conservative dose escalation, dose modification, and by the use of growth factors if necessary. Prophylactic and therapeutic treatments against skin and mucosal toxicity and therapeutic recommendations in case of injection site reactions can be used as needed.

The trial can be conducted as an open-label, phase 1, multicenter, dose escalation trial of TH-302 combined with gemcitabine and nab-paclitaxel in previously untreated subjects with metastatic or locally advanced unresectable pancreatic adenocarcinoma. The trial can investigate safety and tolerability, and define the MTD of TH-302 combined with gemcitabine and nab-paclitaxel. In the trial, TH-302 is administered 2 to 2.5 hours prior to administration of nab-paclitaxel and then gemcitabine, a schedule based on enhanced antitumor efficacy observed in preclinical studies using similar schedules.

The trial will consist of two parts, a dose escalation part and a cohort expansion part at MTD. The dose escalation part will have a 3+3 design and will include subjects with metastatic or locally advanced unresectable pancreatic adenocarcinoma. After the MTD or recommended Phase 2 dose (RP2D) is confirmed in the dose escalation part, the cohort expansion part will be conducted in at least 15 additional subjects. Two RP2D doses may be investigated.

The trial will be conducted in three phases: screening, treatment, and follow-up. Screening assessments will be conducted within 21 days prior to Cycle 1 Day 1. Study drugs will be administered in successive 28-day cycles until there is evidence of progressive disease according to RECIST intolerable toxicity, or the subject discontinues the study drugs for other reasons. Upon discontinuation from the study drugs, an end of treatment visit will be conducted followed by a post-treatment safety visit either 30 days (±3 days) after the last administration of study drugs or immediately before the administration of a new cancer therapy.

Consented subjects who meet the eligibility criteria will be assigned by the sponsor or designee to a TH-302 dose escalation cohort. All screening evaluations will be completed within 21 days prior to the first TH-302 administration. After the subject gives written, informed consent the following evaluations will be performed: complete physical examination, assessment of weight, vital signs measurements (blood pressure, heart rate (HR), respiratory rate, temperature), demographic data recording, 12-lead ECG, Eastern Cooperative Oncology Group (ECOG) performance status, medical history recording, cancer history recording, medication history recording, standard laboratory tests (hematology, biochemistry, urinalysis), pregnancy test in women of childbearing, potential, baseline tumor imaging (computerized tomography (CT) or magnetic resonance imaging (MRI) in the dose escalation part and [¹⁸F]-FDG PET in the cohort expansion at MTD part), sample collection for tumor marker cancer antigen 19-9 (CA19-9), sample collection for serum and plasma hypoxia markers, and assessment of eligibility.

[¹⁸F]-FDG PET metabolic response provides the most sensitive and earliest assessment of tumor response and facilitates comparison of the trial regimen with the gemcitabine and nab-paclitaxel regimen. [¹⁸F]-FDG PET imaging to assess the effect of the trial regimen on tumor metabolic activity will be assessed only in subjects treated in the cohort expansion at the MTD. The PK endpoints will provide additional PK data necessary for the overall assessment of the feasibility of the combination of TH-302 with gemcitabine and nab-paclitaxel.

Analysis of genetic variants of genes, such as but not limited to CYP2C9, CYP2D6, and CYP2C19, that may potentially influence the PK of TH-302. and Br-IPM, can be performed. Tumor DNA may also be isolated from tumor tissue samples to explore genetic variants such as BRCA1 and BRCA2 mutations that may be associated with efficacy of TH-302. Genetic variants associated with the PK, safety, or efficacy of gemcitabine and nab-paclitaxel may also be explored. A separate consent will be requested.

For inclusion in the trial, all of the following inclusion criteria must be fulfilled: at least 18 years of age; able to understand the purposes and risks of the trial and sign a written informed consent form approved by the site's Institutional Review Board (IRB)/Independent Ethics Committee (IEC); locally advanced unresectable or metastatic pancreatic adenocarcinoma proven by histology or cytology and previously untreated with chemotherapy or systemic therapy other than: radiosensitizing doses of 5-fluorouracil, determined according to standard practice, radiosensitizing doses of gemcitabine if relapse occurred at least 6 months after completion of gemcitabine, determined according to standard practice, neoadjuvant chemotherapy if relapse occurred at least 6 months after surgical resection; and adjuvant chemotherapy if relapse occurred at least 6 months after completion of adjuvant chemotherapy; documentation of disease progression since any prior therapy, as judged by the investigator; ECOG performance status of 0 or 1; life expectancy of at least 3 months; acceptable liver function: bilirubin≦1.5 times the upper limit of normal (ULN); does not apply to subjects with Gilbert's syndrome, aspartate aminotransferase (AST) and alanine aminotransferase (ALT)≦3 times the ULN; if liver metastases are present, then≦5 times the ULN is allowed; acceptable renal function: serum creatinine≦1.5 times the ULN or calculated creatinine clearance≧60 mL/min (by the Cockcroft-Gault formula); acceptable hematologic status (without growth factor support or transfusion dependency): absolute neutrophil count≧1500 cells/μL, platelet count≧150000/μL, and hemoglobin≧9.0 g/dL; QTc interval calculated according to Bazett's formula (QTc=QT/RR^{0.5}; RR=RR interval) of ≦450 msec on screening ECG; and only subjects with metastatic pancreatic adenocarcinoma who have measurable disease according to RECIST 1.1 criteria should be enrolled for the cohort expansion at MTD.

The treatment phase will consist of 28-day cycles. On Days 1, 8, and 15 of each cycle, TH-302 will be administered i.v. 2 to 2.5 hours prior to the i.v. administration of nab-paclitaxel at 100 mg/m² followed by the i.v. administration of gemcitabine at 800 mg/m². Doses of nab-paclitaxel at 125 mg/m² and gemcitabine at 1000 mg/m² may be administered as part of the final determination of a RP2D. In addition, gemcitabine, nab-paclitaxel, and TH-302 doses can be delayed or modified or both for hematological and non-hematological toxicity. The combination treatment will continue until disease progression according to RECIST 1.1, intolerable toxicity, or subject discontinuation of study drug for other reasons.

The evaluations that will be performed at specified time points are: limited physical examination, update of medical history, assessment of weight, vital signs measurements (blood pressure, HR, respiratory rate, temperature), 12-lead ECGs, ECOG performance status, standard laboratory tests (hematology, biochemistry, urinalysis), PK sampling, sample collection for tumor marker (CA19-9), sample collection for serum and plasma hypoxia markers, pregnancy test in women of childbearing potential, tumor imaging (CT or MRI in the dose escalation part and [¹⁸F]-FDG PET in the cohort expansion at MTD part), sample collection for pharmacogenomics testing, concomitant medication recording, and AE assessment. If the subject has consented, a tumor tissue sample will be obtained from a prior tumor resection or biopsy.

Three dose levels of TH-302 are planned (170, 240, 340 mg/m²) in cohorts of 3 to 6 subjects each. Initially, 3 subjects will be enrolled and dosed in each dose escalation cohort. If a subject experiences a DLT, 3 additional subjects will be enrolled at that dose level (a total of 6 subjects in the cohort). If no additional DLTs are observed, the dose escalation will continue in the next cohort. However, if 2 or more of the 6 subjects within a cohort experience a DLT, that dose will be considered to have exceeded the MTD. The MTD will then be defined at the highest dose level at which 6 subjects were treated and 1 or none of the subjects experienced a DLT. If significant differences in toxicity are observed between the intolerable dose (highest administered dose) and the prior dose level, an intermediate dose level may be investigated.

At least 15 additional subjects, with metastatic disease, will be enrolled in the cohort expansion at MTD/RP2D. This cohort will receive the MTD or RP2D determined in the dose escalation part to further evaluate the safety, tolerability, and preliminary antitumor activity of TH-302 in combination with gemcitabine and nab-paclitaxel.

The primary endpoint is the number of subjects experiencing at least 1 DLT within the first treatment cycle (28 days) after the first administration of TH-302. Safety and tolerability endpoints will consist of TEAEs graded according CTCAE version 4.03, SAEs, and deaths. In addition, drug exposure and standard laboratory tests (hematology, biochemistry, urinalysis, and pregnancy test in women of childbearing potential), 12-lead ECGs, physical examinations, and assessment of weight and vital signs will also be performed. Tumor assessment endpoints will consist of overall response, duration of response, and PFS according to RECIST 1.1 criteria, CA19-9 levels, and the effect of the trial regimen on tumor metabolic activity by tumor imaging using PET scans (cohort expansion at MTD part only).

All subjects will be eligible to receive best supportive care defined as any standard supportive measure that is not considered primary treatment of the disease under study. Use of growth factors for the treatment of myelosuppression will be according to the American Society of Clinical Oncology (ASCO) guidelines should they prove necessary. The triple agent combination treatment will continue until disease progression, intolerable toxicity, or subject discontinuation (e.g., withdrawal of consent) occurs. Following discontinuation of gemcitabine or nab-paclitaxel or both, subjects will be eligible to continue treatment with either chemotherapy agent in combination with TH-302 or single agent TH-302 if the subject is deriving clinical benefit in the opinion of the investigator and vice versa (continuation of gemcitabine or nab-paclitaxel in cases of TH-302 toxicity). The dose of TH-302 in these subjects will be the one assigned at Cycle 1 or lower if modified in response to toxicities. No intra-subject TH-302 dose escalation will be allowed. Subjects, for whom the TH-302 dose was previously reduced due to toxicity, will continue to receive the reduced dose of TH-302.

TH-302 (concentrate for solution for administration) for use in the trial is a sterile liquid formulation of TH-302. TH-302 is formulated with 70% ethanol anhydrous, 25% dimethlyacetamide, and 5% polysorbate 80. It will be supplied by the sponsor in a 10-mL glass vial with a rubber stopper and flip-off seal. TH-302 drug product is a clear, colorless to light yellow solution, essentially free of visible particulates. Each single use vial contains a nominal fill volume of 6.5 mL of TH-302 drug product for a nominal total of 650 mg of TH-302 (corresponds to 100 mg/mL) and will be labeled clearly, disclosing the lot number, route of administration, required storage conditions, sponsor's name, and appropriate precautionary labeling as required by applicable regulations. Dilution prior to administration is required per the pharmacy manual.

TH 302 drug product is supplied in a 10 mL glass vial and will be diluted prior to administration with commercially available 5% dextrose in water to a total volume of 500 mL (1000 mL for total dose of ≧1000 mg) per administration to obtain the desired final concentration. Each dose of TH 302 will be prepared in a non di(2 ethylhexyl)phthalate (non DEHP), containing 5% dextrose in water, and administered i.v. via a non DEHP containing i.v. administration set. The starting dose of TH 302 will be 170 mg/m². Two additional dose levels are planned (240 and 340 mg/m²). Additional intermediate dose levels may be investigated to manage emerging toxicities. TH 302 will be administered via i.v. administration over 30 minutes on Days 1, 8, and 15 of every 28-day cycle.

The body surface area (BSA) should be recalculated and the dose adjusted on each dosing occasion. Body surface area should be calculated based on a standard formula, such as the Mosteller formula: BSA(m²)([height(cm)×weight(kg)]/3600)^{1/2} e.g., BSA=square root((cm*kg)/3600) or in inches and pounds: BSA (m²)=([height(in)×weight(lbs)]/3131)^{1/2}.

Each TH 302 dose will be administered as a 500 mL (1000 mL for total dose of ≧1000 mg) volume that should be given i.v. over 30 minutes. Longer durations of administration are permitted based on the investigator's judgment of the time required to administer the dose.

Supravenous erythema and hyperpigmentation have been reported at the injection site; severe cellulitis, vessication, and tissue necrosis may occur if extravasation of TH 302 occurs during administration. Care in the administration of TH 302 will reduce the chance of perivenous infiltration. If any signs or symptoms of extravasation occur, the administration of TH 302 should be immediately terminated and restarted in another vein. TH 302 should always be administered via a freely flowing i.v. line, preferably, where feasible, through a central venous catheter. Administration through small veins, particularly on the hands and feet is discouraged. Because of the progressive nature of extravasation reactions, close observation and plastic surgery consultation is recommended.

Prophylaxis against nausea and vomiting should be implemented using a regimen intended for moderately emetogenic chemotherapy.

TH 302 administration reactions have been observed. These reactions have been characterized by lip swelling and urticaria that responded to steroid and antihistamine treatment. It is recommended that a steroid such as dexamethasone (or equivalent) be included in the antiemetic regimen prior to administration. Symptoms and signs of hypersensitivity include fever, myalgia, headache, rash, pruritus, urticaria, angioedema, chest discomfort, dyspnea, coughing, cyanosis, and hypotension. If the nature and the severity of the reaction require termination of treatment, it should be determined if the reaction may or may not be an immunoglobulin E mediated process. If there are symptoms such as upper airway obstruction or hypotension that suggest anaphylaxis or an anaphylactoid reaction, then treatment with an antihistamine (e.g., diphenhydramine 25 to 50 mg oral, intramuscular, or slow i.v., or equivalent) and low dose steroid (e.g., hydrocortisone, 100 mg i.v. or equivalent) should be considered by the investigator as appropriate. If the event is clearly anaphylaxis, then epinephrine ( 1/1000, 0.3 to 0.5 mL given subcutaneously or equivalent) should be considered as well as standard treatment approaches. In the case of bronchospasm, inhaled β-agonist should be considered. Idiosyncratic reaction may also be treated with an antihistamine and low dose steroids depending on their severity. Reactions to the administration of TH 302 should be assessed and treated in a similar manner. For all reactions to TH 302, the investigator should consult with the Medical Monitor to determine the appropriate course of action for future treatment.

Nab-paclitaxel will be administered intravenously over 30 minutes on Days 1, 8, and 15 of every 28-day cycle at doses of 100-125 mg/m². The BSA should be recalculated and the dose adjusted on each dosing occasion. Nab-paclitaxel dose modifications will be conducted according to guidelines, The nab-paclitaxel administration should start 2 to 2.5 hours after completion of the TH-302 administration. Nab-paclitaxel can be purchased from commercially available sources. Nab-paclitaxel will be reconstituted and diluted prior to administration with 0.9% sodium chloride (without preservatives) accordance with its product labeling.

Gemcitabine will be administered via i.v. administration over 30 minutes on Days 1, 8, and 15 of every 28-day cycle at doses of 800-1000 mg/m². The BSA should be recalculated and the dose adjusted on each dosing occasion. Gemcitabine dose modifications will be conducted according to guidelines. The gemcitabine administration should start after the nab-paclitaxel administration. Gemcitabine can be from commercially available sources. Gemcitabine will be reconstituted and diluted prior to administration with 0.9% sodium chloride (without preservatives) in accordance with its product labeling.

Prophylactic granulocyte colony-stimulating factor (G-CSF) may be implemented if it is observed that neutropenia results in dose reduction or dose delays, as suggested in the ASCO Guidelines. Use of hematopoietic colony-stimulating factors is permitted following the ASCO guidelines. Based on prior experience with this regimen, in the event a subject experiences neutropenia, and G-CSF is initiated, and neutropenia recovers within 48 hours after beginning treatment with G-CSF, G-CSF may be implemented in the management of neutropenia to avoid dose reductions or holding a dose. Hemoglobin must be ≧9 g/dL at Cycle 1 Day 1 and must be ≧8 g/dL for all subsequent doses. If hemoglobin is <8 g/dL then appropriate measures according to standard clinical practice must be taken prior to any further dose administration.

Any medications (other than those excluded by the protocol) that are considered necessary for the subjects' welfare and will not interfere with the study drugs may be given at the investigator's discretion. Female subjects who have been on hormone replacement therapy for menopausal symptoms for a period of at least 2 months will not be excluded from the trial provided the hormone replacement therapy regimen remains unchanged during the conduct of the trial. Prophylactic hematopoietic colony-stimulating factors may be implemented in subsequent cycles if neutropenia results in dose reduction or dose delay at prior doses. Therapeutic use of hematopoietic colony stimulating factors is permitted following ASCO guidelines.

Prophylaxis against nausea and vomiting should be implemented using a regimen intended for moderately emetogenic chemotherapy. Inclusion of dexamethasone (or equivalent) in the antiemetic regimen is recommended. Prophylactic and therapeutic recommendations against skin and mucosal toxicity and therapeutic recommendations in case of injection site reactions may be taken as described above.

Palliative radiotherapy to non-target lesions is permitted if it becomes necessary while the subjects are in the trial. Prophylactic and therapeutic steps against skin and mucosal toxicity and therapeutic treatment in case of injection site reactions is permissible.

The follow-up phase will consist of 2 visits, an end of treatment visit and a safety visit. The end of treatment visit will be conducted within 1 week after discontinuation of the study drug, treatment or immediately before initiation of any other anti-cancer therapy, whichever occurs first. The safety visit will be conducted either 30 (±3) days after the last administration of study drug or immediately before initiation of any other anti-cancer therapy. Upon discontinuation from the treatments (end of treatment visit), the subject will have a complete physical examination, assessment of weight, ECOG performance status, vital signs measurements (blood pressure, HR, respiratory rate, temperature), 12-lead ECG, standard laboratory tests (hematology, biochemistry, urinalysis), sample collection for tumor marker (CA19-9), sample collection for serum and plasma hypoxia markers, pregnancy test for women of childbearing potential, tumor imaging (CT or MRI, only required if not performed within the past 8 weeks and if clinically appropriate), and concomitant medication recording, and AEs assessment.

A safety visit will be conducted either 30 days (±3 days) after the last administration of study drugs or immediately before initiation of any other cancer therapy. Information an further lines of therapy will be collected. The evaluations performed at the safety visit are: complete physical examination, vital signs assessments (blood pressure, HR, respiratory rate, temperature), standard laboratory tests (hematology, biochemistry, urinalysis), and pregnancy test for women of childbearing potential. Subjects will be contacted for AEs and subsequent cancer therapy information every 3 months for a minimum of 12 months.

For all purposes in the United States of America, each and every publication and patent document cited herein is incorporated herein by reference for all purposes as if each such publication or document was specifically and individually indicated to be incorporated herein by reference.

While the invention has been described with reference to the specific embodiments, changes can be made and equivalents can be substituted to adapt to a particular context or intended use, thereby achieving benefits of the invention without departing from the scope of the claims that follow.

1. A combination of pharmaceutical agents comprising a hypoxia-activated prodrug and a taxane for simultaneous or sequential use in the treatment of pancreatic cancer.

2. The combination of claim 1, further comprising a nucleoside analog chemotherapeutic.

3. A method of treating pancreatic cancer, comprising simultaneously or sequentially administering an effective combination of pharmaceutical agents comprising a hypoxia-activated prodrug and a taxane.

4. The method of claim 3, further comprising administering a nucleoside analog chemotherapeutic.

5. The method of claim 3, comprising administering a hypoxia-activated prodrug and a taxane, but not a nucleoside analog.

6. (canceled)

7. The method, of claim 3, wherein the hypoxia-activated prodrug has the structure according to Formula (I): 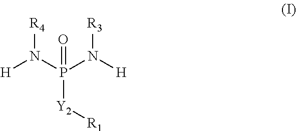
wherein Y₂ is O, S, NR₆, NCOR₆, or NSO₂R₆;
wherein R₆ is C₁-C₆ alkyl, C₁-C₆ heteroalkyl, aryl, or heteroaryl; R₃ and R₄ are independently selected from 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl; and R₁ has the formula L-Z₃;
wherein L is C(Z₁)₂; each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl; or L is: 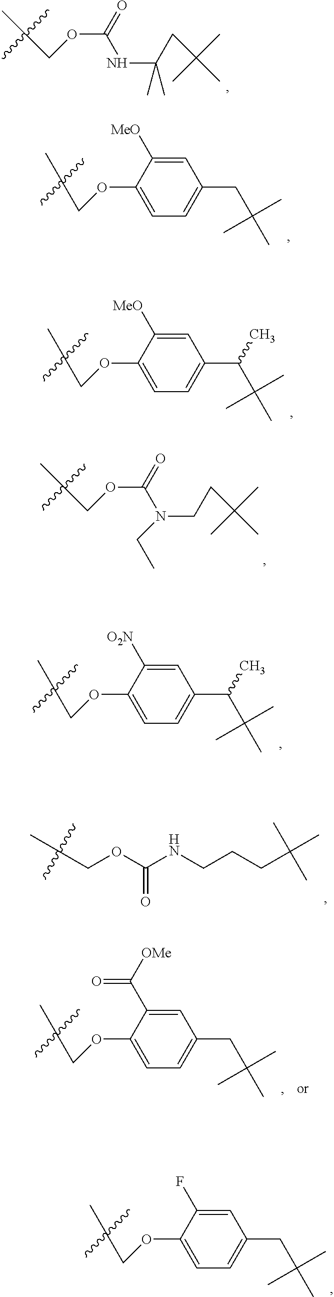
wherein Z₃ is a bioreductive group having a formula selected from: 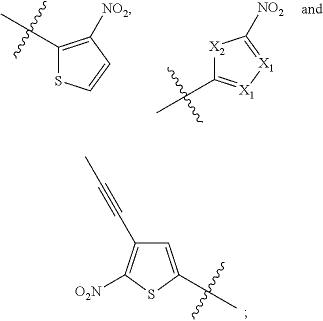
wherein each X₁ is independently N or CR₈; X₂ is NR_{S}, S, or O; each R₇ is independently C₁-C₆ alkyl, C₁-C₆ heteroalkyl, C₃-C₈ cycloalkyl, heterocyclyl, aryl or heteroaryl; and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl;
or a pharmaceutically acceptable salt thereof.

8. The method of claim 7, wherein the hypoxia-activated prodrug is TH-302.

9. The method of claim 3, wherein the taxane is a 9-dihydrotaxol analog having a chemical structure according to Formula II: 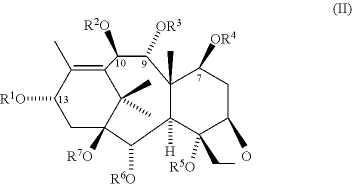
wherein R₂, R₄, R₅ and R₇ in formula (II) are independently hydrogen, alkyl, alkanoyl or aminoalkanoyl. R₃ in formula (II) is hydrogen, alkyl or aminoalkanoyl. R₆ in formula (II) is hydrogen, alkyl, alkanoyl, aminoalkanoyl or phenylcarbonyl (—C(O)-phenyl);
wherein R₁ has the following structure: 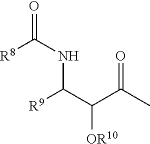
wherein R₈ is hydrogen, alkyl, phenyl, substituted phenyl, alkoxy, substituted alkoxy, amino, substituted amino, phenoxy or substituted phenoxy; R₉ is hydrogen, alkyl, hydroxyalkyl, alkoxyalkyl, aminoalkyl, phenyl or substituted phenyl; and R₁₀ is hydrogen, alkanoyl, substituted alkanoyl or aminoalkanoyl.

10. The method of claim 9, wherein the taxane is paclitaxel or nab-paclitaxel.

11. The method of claim 3, wherein the combination of pharmaceutical agents comprises a nucleoside analog chemotherapeutic that has a chemical structure according to Formula III: 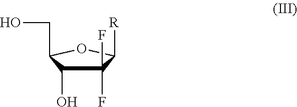
wherein R is a nitrogen-containing monocyclic or heterocyclic aromatic compound selected so that the compound may mimic a nucleoside analog and block enzymatic reactions associated with DNA biosynthesis.

12. The method of claim 11, wherein R is selected from the following: 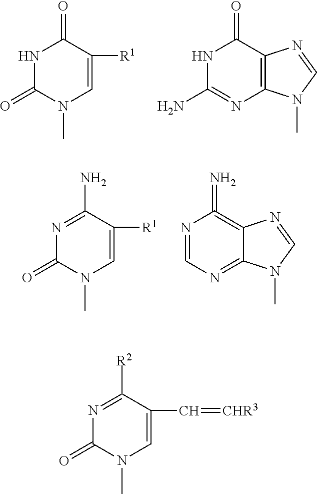
wherein R₁ is hydrogen, methyl, bromo, fluoro, chloro or iodo; and R₂ is hydroxy; and R₃ is hydrogen, bromo, chloro or iodo.

13. The method of claim 11, wherein the nucleoside analog chemotherapeutic is gemcitabine.

14. The method of claim 3, wherein the hypoxia-activated prodrug is administered at least about 30 minutes before the taxane and/or at least about 30 minutes before the nucleoside analog chemotherapeutic.

15. (canceled)

16. The method of claim 14, wherein the hypoxia-activated prodrug, the taxane, and optionally the nucleoside analog chemotherapeutic are administered in a plurality of cycles, each comprising consecutively administering each of said drugs at least once a week for three consecutive weeks, followed by one week in which none of said drugs is administered.

17. The method of claim 3, wherein the hypoxia-activated prodrug is TH-302, the taxane is nab-paclitaxel, and the nucleoside analog is gemcitabine.

18. (canceled)
